(19) United States
(12) Patent Application Publication
(10) Pub. No.: US 2025/0249254 A1
(43) Pub. Date: Aug. 7, 2025

(54) SYSTEMS AND METHODS FOR SCHEDULED NEUROMODULATION THERAPY

(71) Applicant: Boston Scientific Neuromodulation Corporation, Valencia, CA (US)

(72) Inventors: Jessica Block, Los Angeles, CA (US); Tianhe Zhang, Studio City, CA (US); Jianwen Gu, Richmond, TX (US); Satya Venkata Sandeep Avvaru, Houston, TX (US)

(21) Appl. No.: 19/022,292

(22) Filed: Jan. 15, 2025

Related U.S. Application Data
(60) Provisional application No. 63/550,918, filed on Feb. 7, 2024.

(51) Int. Cl. A61N 1/36; G16H 10/20; G16H 20/40

(52) U.S. Cl. CPC A61N 1/36139; A61N 1/36021; A61N 1/36132; A61N 1/36167; G16H 10/20; G16H 20/40

(57) ABSTRACT

Systems and methods for schedule neuromodulation therapy are disclosed. An exemplary system comprises an electrostimulator and a controller. The electrostimulator can be configured to provide electrostimulation to a neural target of a patient. The controller circuit can be operably connected to the electrostimulator. The controller circuit can be configured to determine an activity parameter based on a schedule parameter of the patient, wherein the schedule parameter is associated with a schedule of the patient; determine stimulation parameters based on the activity parameter; and deliver the electrostimulation to the neural target of the patient using the stimulation parameters.

### CLAIM OF PRIORITY

This application claims the benefit of U.S. Provisional Application No. 63/550,918, filed on Feb. 7, 2024, which is hereby incorporated by reference in its entirety.

### TECHNICAL FIELD

This document relates generally to medical systems, and more particularly, but not by way of limitation, to systems, devices, and methods for scheduled neuromodulation therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

A therapy device may be configured to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS. PNS or TENS), may be configured to treat pain. In another nonlimiting example, a device, such as a myocardial stimulator and/or neurostimulator, may be configured to treat cardiovascular condition. Settings of the therapy device may be programmed based on observed clinical effects so that the therapy provides desirable intended effects (e.g., reduced tremor, bradykinesia, and dyskinesia for a PD therapy, desirable pain relief or paresthesia coverage for a pain therapy, desirable blood pressure and/rhythms for a cardiovascular therapy) while avoiding undesirable side effects.

### SUMMARY

An example (e.g., “Example 1”) of a system may include an electrostimulator configured to provide electrostimulation to a neural target of a patient; and a controller circuit operably connected to the electrostimulator, the controller circuit configured to: determine an activity parameter based on a schedule parameter of the patient, wherein the schedule parameter is associated with a schedule of the patient; determine stimulation parameters based on the activity parameter; and deliver the electrostimulation to the neural target of the patient using the stimulation parameters.

In Example 2, the subject matter of Example 1 optionally includes wherein the schedule parameter is an upcoming schedule parameter for delivering the electrostimulation during a subsequent time period.

In Example 3, the subject matter of any one or more of Examples 1-2 optionally include wherein the controller circuit is configured to determine the schedule parameter by accessing the schedule on a computing device of the patient.

In Example 4, the subject matter of Example 3 optionally includes wherein the controller circuit is configured to update the schedule parameter based on a change in the schedule and is configured to determine the activity parameter based on the updated schedule parameter.

In Example 5, the subject matter of any one or more of Examples 1˜4 optionally include wherein the controller circuit is configured to determine the schedule parameter by receiving input data from the patient associated with the schedule parameter.

In Example 6, the subject matter of any one or more of Examples 1-5 optionally include wherein the controller circuit is configured to determine a plurality of schedule parameters and select the schedule parameter from the plurality of schedule parameters based on which period of time the schedule parameter is associated with.

In Example 7, the subject matter of any one or more of Examples 1-6 optionally include wherein the activity parameter comprises at least one of sleeping, reading, working, performing errands, leisure activity, exercising, sitting in a vehicle, and performing daily chores.

In Example 8, the subject matter of any one or more of Examples 1-7 optionally include wherein the schedule parameter comprises a time of day of a day of a week and the activity parameter is determined based on the time of day and the day of the week.

In Example 9, the subject matter of any one or more of Examples 1-8 optionally include wherein the controller circuit is configured to determine the activity parameter by using: a first set of schedule data received from the patient; and a second set of schedule data based on a time of day and week.

In Example 10, the subject matter of Example 9 optionally includes wherein the controller circuit is configured to receive the first set of schedule data by sending a request to the patient for data.

In Example 11, the subject matter of any one or more of Examples 9-10 optionally include wherein the controller circuit is configured to analyze the second set of schedule data to determine whether a particular activity during a particular period of time deviates from a normal routine of the patient.

In Example 12, the subject matter of Example 11 optionally includes wherein the controller circuit is configured to request additional data from the patient in response to determining that the particular activity deviates from the normal routine.

In Example 13, the subject matter of Example 12 optionally includes wherein at least one of the first set of schedule data, the second set of schedule data, or the additional data is used to adjust the stimulation parameters.

In Example 14, the subject matter of any one or more of Examples 1-13 optionally include wherein the controller circuit is configured to: detect a change in GPS location or time zone experienced by the patient; and request travel data from the patient based on the detected change in time zone.

In Example 15, the subject matter of Example 14 optionally includes wherein the travel data indicates at least one of a reason for travel of the patient or expected activities while traveling; and the controller circuit is configured to adjust the stimulation parameters based on the travel data.

Example 16 includes subject matter (such as a method, means for performing acts, machine readable medium including instructions that when performed by a machine cause the machine to perform acts, or an apparatus to perform). The subject matter may include determining an activity parameter associated with providing electrostimulation to a neural target of a patient based on a schedule parameter associated with the patient, wherein the activity parameter is determined based on received activity data from the patient. The subject matter may include determining stimulation parameters based on the activity parameter. The subject matter may include delivering the electrostimulation to the neural target of the patient via a stimulating electrode using the stimulation parameters.

In Example 17, the subject matter of Example 16 optionally includes wherein the schedule parameter is determined based on a schedule of the patient.

In Example 18, the subject matter of Example 17 optionally includes wherein the schedule parameter is determined by accessing the schedule on a computing device of the patient.

In Example 19, the subject matter of any one or more of Examples 17-18 optionally include updating the schedule parameter based on a change in the schedule.

In Example 20, the subject matter of any one or more of Examples 16-19 optionally include wherein the activity data is received from the patient in response to sending a request to the patient for the activity data.

In Example 21, the subject matter of any one or more of Examples 16-20 optionally include wherein the schedule parameter is an upcoming schedule parameter for delivering the electrostimulation during a subsequent time period.

In Example 22, the subject matter of any one or more of Examples 16-21 optionally include analyzing the activity parameter and the time of day and the day of the week associated with the activity parameter to determine whether the activity parameter is a deviation from a normal routine of the patient.

In Example 23, the subject matter of Example 22 optionally includes requesting additional data from the patient in response to determining that the activity parameter deviates from the normal routine.

In Example 24, the subject matter of any one or more of Examples 16-23 optionally include wherein determining stimulation parameters comprises determining a period of time to deliver the electrostimulation using the stimulation parameters.

In Example 25, the subject matter of Example 24 optionally includes wherein the period of time is determined based on a corresponding period of time that the activity parameter is associated with.

In Example 26, the subject matter of any one or more of Examples 16-25 optionally include wherein determining the stimulation parameters comprises: determining a first set of stimulation parameters used to deliver the electrostimulation during a first period of time; and determining a second set of stimulation parameters used to deliver the electrostimulation during a second period of time.

In Example 27, the subject matter of Example 26 optionally includes wherein the first period of time is associated with a first activity parameter and the second period of time is associated with a second activity parameter.

In Example 28, the subject matter of any one or more of Examples 16-27 optionally include determining a therapy satisfaction experienced by the patient during a period of time.

In Example 29, the subject matter of Example 28 optionally includes wherein the stimulation parameters are determined using the determined pain level as an input.

In Example 30, the subject matter of any one or more of Examples 28-29 optionally include wherein the pain level is determined by receiving pain data from the patient.

In Example 31, the subject matter of Example 30 optionally includes wherein the pain data is received in response to sending a request to the patient to indicate the pain level.

Example 32 includes subject matter (such as a method, means for performing acts, machine readable medium including instructions that when performed by a machine cause the machine to perform acts, or an apparatus to perform). The subject matter may include determining an activity parameter and a schedule parameter associated with providing electrostimulation to a neural target of a patient by accessing a schedule of the patient. The subject matter may include determining stimulation parameters based on the activity parameter. The subject matter may include delivering the electrostimulation to the neural target of the patient via a stimulating electrode using the stimulation parameters.

In Example 33, the subject matter of Example 32 optionally includes wherein the instructions are executable by the processor to determine a pain level at the time of day and the day of the week for the patient.

In Example 34, the subject matter of any one or more of Examples 32-33 optionally include wherein the instructions are executable by the processor to determine the stimulation parameters such that a prior electrostimulation delivered during a similar activity at a similar time and day of week that achieved a particular therapy level is scheduled to be delivered during the similar activity when anticipated to be performed at a later period of time.

In Example 35, the subject matter of any one or more of Examples 32-34 optionally include wherein the instructions are executable by the processor to adjust the determined stimulation parameters in response to receiving particular pain level data from the patient associated with the determined stimulation parameter.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver SCS and/or DBS.

FIG. 2 illustrates, by way of example and not limitation, an implantable pulse generator (IPG) in an SCS and/or DBS system.

FIGS. 3A-3B illustrate, by way of example and not limitation, leads that may be coupled to the IPG to deliver electrostimulation such as SCS and/or DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates, by way of example and not limitation, a method of scheduled neuromodulation therapy.

FIG. 8 illustrates, by way of example and not limitation, a method of scheduled neuromodulation therapy.

FIG. 9 illustrates, by way of example and not limitation, a diagram of activities during a daily schedule of a patient.

FIG. 10 illustrates, by way of example and not limitation, a diagram of schedules that include activities for different types of days of a patient.

FIG. 11 illustrates, by way of example and not limitation, a diagram of a calendar showing the different types of days for a patient.

FIG. 12 illustrates, by way of example and not limitation, an example of activity parameters of a patient.

FIG. 13 illustrates, by way of example and not limitation, a neuromodulation therapy system, which may be used to deliver SCS and/or DBS.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

There may be differing effectiveness of neuromodulation therapy depending on a patient's state, e.g., vary depending on a medication level, a physical state (sleep, awake, physical activity, etc.), condition or disorder, anatomy (anatomical target), or symptoms to improve (e.g., tremor to cognitive skill improvement). These states may be affected by a schedule of the patient and corresponding activities based on that schedule. States may also include an active state vs. inactive state, an ON or OFF state, low state, high state, optimal states, inflection points in state, stable states, transitioning from one state to another, etc. These states may be correlated to particular, or significant, events. The particular events may be used to normalize data recordings or to indicate optimal times for testing and/or providing therapy. Further, adjusting the neuromodulation therapy to these schedules and corresponding conditions may provide for more effective treatment.

A schedule or schedules of a patient can be used to plan out neuromodulation therapy over a period of time, over a period of days, weeks, etc., based on the state of the patient during the scheduled activities. The state of the patient can be inferred from a patient's daily calendar, data input by the patient about planned activities, etc. The patient's calendar can also provide clock time information associated with each of the activities and therefore states of the patient and their durations. The neuromodulation therapy can be administered based on clock time, patient activity during a time interval, and patient preferences. The clock time could be taken from a patient device and/or from information sent to or from the patient device. Further, patient activity, pain levels, and/or other biometric ranges may be provided as input to determine which neuromodulation therapy to provide during which time intervals.

By providing an anticipated schedule and corresponding activities, the neuromodulation therapy can be planned and adjusted accordingly. The schedule may be adjusted in real time and therefore the planned neuromodulation therapy adjusted as well. Features of the sensed signals may vary in relation to a state of the patient for each associated activity and may be used to estimate a schedule for administering medication, to estimate medication efficacy, and/or for administering electrostimulation therapy.

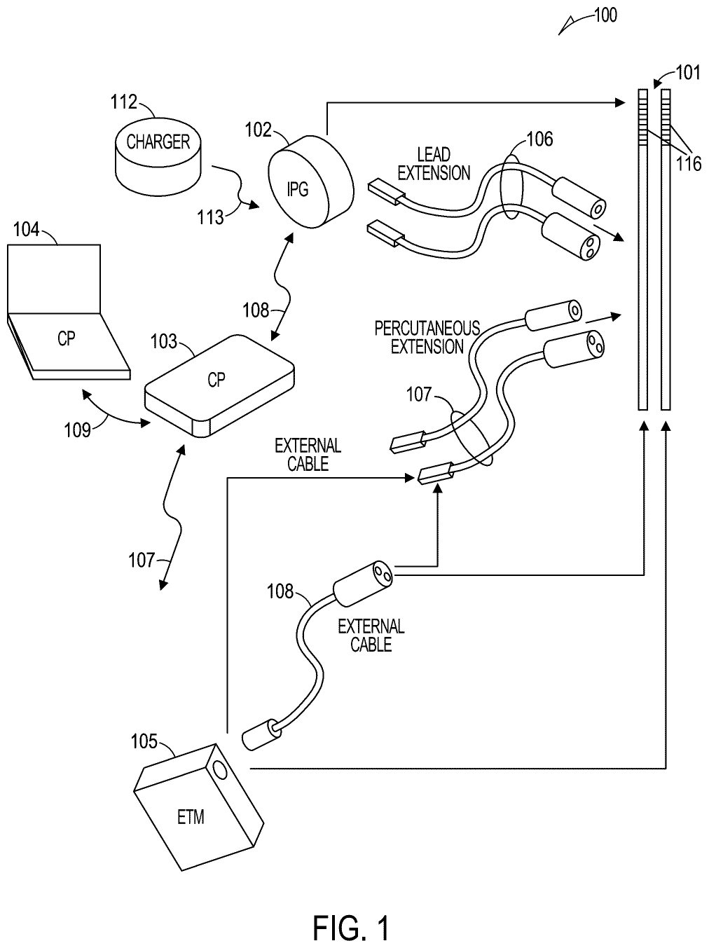

FIG. 1 illustrates, by way of example and not limitation, an electrical stimulation system 100, which may be used to deliver SCS and/or DBS. The electrical stimulation system 100 may generally include one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads may be provided, including only one, as long as the number of electrodes is greater than two (including the IPG case function as a case electrode) to allow for lateral steering of the current. Alternatively, a surgical paddle lead may be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 may likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters may be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies may be performed by executing software instructions contained within the CP 104. Alternatively, such programming methodologies may be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

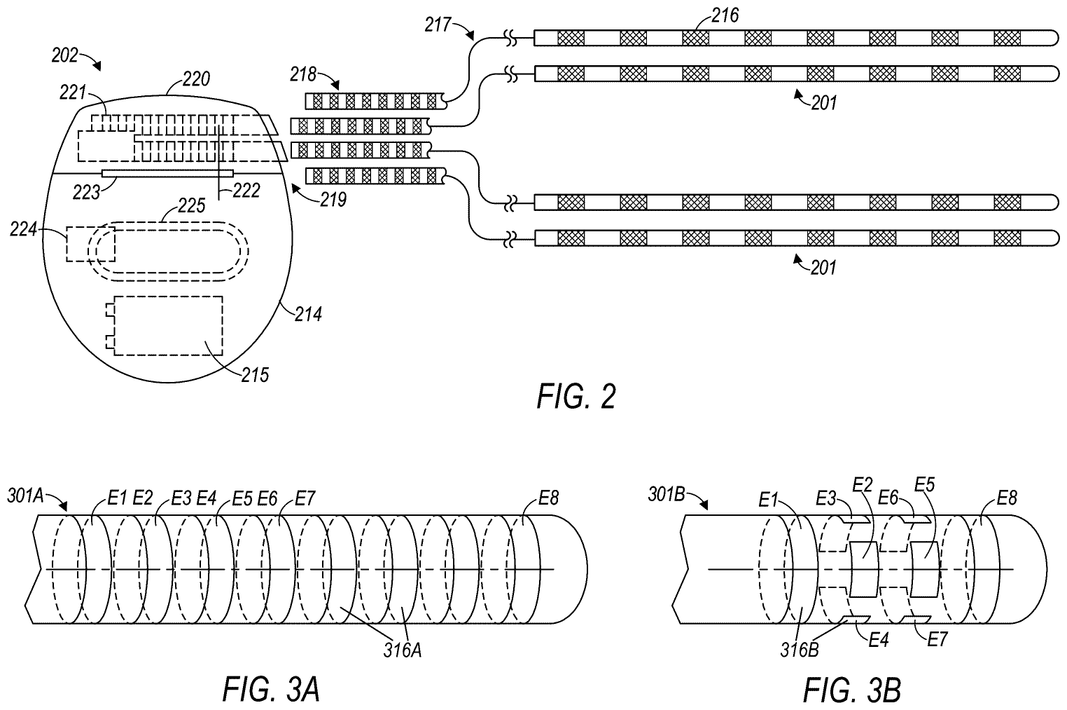

FIG. 2 illustrates, by way of example and not limitation, an IPG 202 in an SCS and/or DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more rows of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore may vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue; a comparison of a set threshold value; user-determined values or thresholds, or a change/delta from a prior measurement. Each of the electrodes may either be used (an active electrode) or unused (OFF). When the electrode is used, the electrode may be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as SCS and/or DBS. FIG. 3A shows a non-directional lead 301A with electrodes 316A disposed at least partially about a circumference of the non-directional lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a directional lead 301B with electrodes 316B including ring electrodes such as E1 at a proximal end and E8 at the distal end. Additionally, the lead 301 also includes a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide more-superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

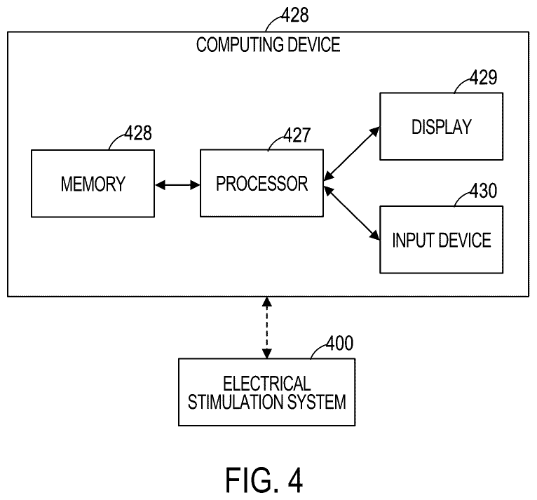

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease.

In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, a closed-loop algorithm for determining or updating stimulation parameters may be implemented in a mobile device, such as a smartphone, which is connected to the IPG or an evaluating device (e.g., a wristband or watch). These devices may also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

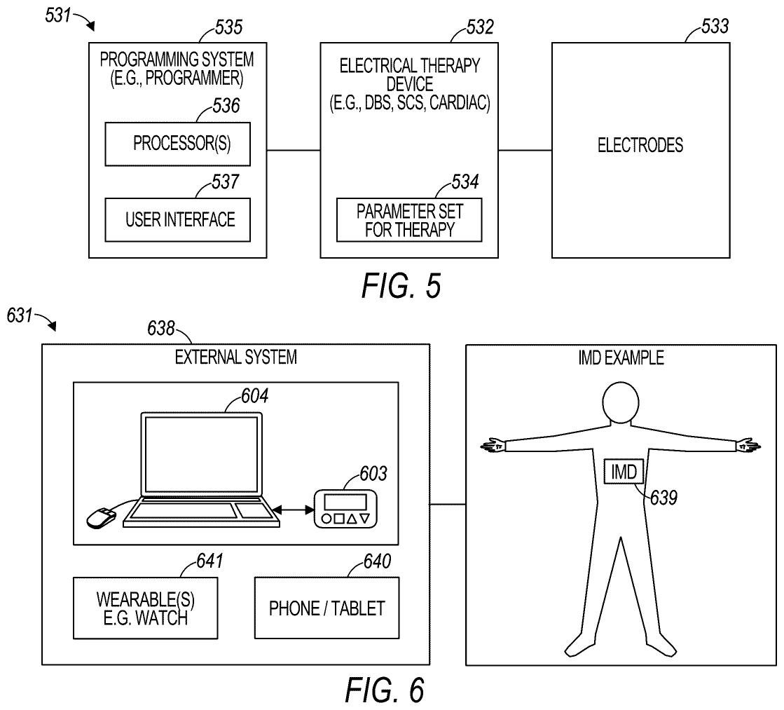

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameter sets through a computerized programming system to allow the optimum modulation parameters to be determined based on patient feedback or other means and to subsequently program the desired modulation parameter sets.

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

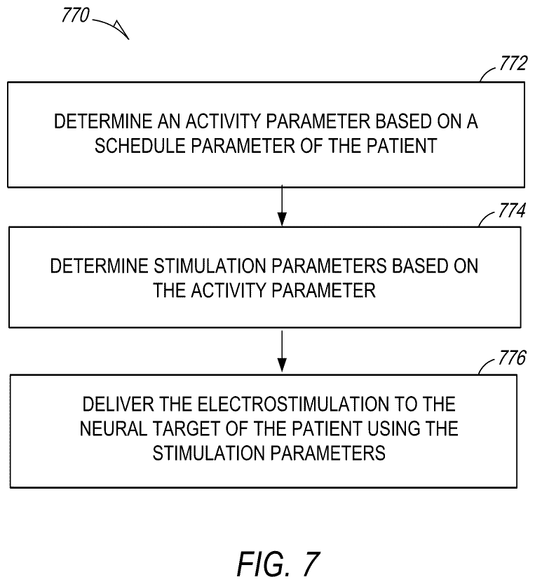

FIG. 7 illustrates, by way of example and not limitation, a method 770 for scheduling neuromodulation therapy. At 772, the method 770 may include determining an activity parameter based on a schedule parameter of the patient. The schedule parameter can be associated with a schedule of the patient. The schedule parameter can be an upcoming schedule parameter for delivering the electrostimulation during a subsequent time period. The schedule parameter can include a time of day or a day of a week. The activity parameter can be determined based on the time of day and the day of the week. The activity parameter can include at least one of sleeping, reading, working, performing errands, leisure activity, exercising, sitting in a vehicle, and performing daily chores.

The activity parameter can be determined by using a first set of schedule data received from the patient and a second set of schedule data based on a time of day and week. The first set of schedule data can be received from the patient in response to sending a request to the patient for data. The second set of schedule data can be analyzed to determine whether a particular activity during a particular period of time deviates from a normal routine of the patient. Additional data can be requested from the patient in response to determining that the particular activity deviates from the normal routine. At least one of the first set of schedule data, the second set of schedule data, or the additional data can be used to adjust the stimulation parameters.

At 774, the method 770 can include determining stimulation parameters based on the activity parameter. Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue; a comparison of a set threshold value; user-determined values or thresholds, or a change/delta from a prior measurement. Each of the electrodes may either be used (an active electrode) or unused (OFF). These and possibly other stimulation parameters taken together comprise a stimulation program that the stimulation circuitry 224 in the IPG 202 of FIG. 2 may execute to provide therapeutic stimulation to a patient.

At 776, the method 770 can include delivering the electrostimulation to the neural target of the patient using the stimulation parameters. An electrostimulator can be used to provide the electrostimulation to the neural target of the patient. A controller circuit can be operably connected to the electrostimulator and be used to perform the steps of method 770.

The method can further include updating the schedule parameter based on a change in the schedule and determining the activity parameter based on the updated schedule parameter. The method 770 can further include determining the schedule parameter by receiving input data from the patient associated with the schedule parameter. The method 770 can further include determining a plurality of schedule parameters and selecting the schedule parameter from the plurality of schedule parameters based on which period of time the schedule parameter is associated with. The method 770 can further include detecting a change in time zone experienced by the patient. In response to detecting the change in time zone, a request for travel data can be sent to the patient. The travel data can indicate at least one of a reason for travel of the patient or expected activities while traveling. The stimulation parameters can be adjusted based on the travel data.

The method 770 may include adjusting a set of stimulation parameters associated with the electrostimulation based on the determined schedule parameter and/or activity parameter. For example, a first set of stimulation parameters may be used to perform electrostimulation in response to the patient being engaged in or about to be engaged in a particular activity parameter that may be associated with a schedule parameter and a second set of stimulation parameters may be used when the patient being engaged in or about to be engaged in a different activity parameter. The set of stimulation parameters may be adjusted to the first set or the second set, or an additional set, based on which activity parameter is associated with the patient during a particular point of the schedule or schedule parameter. The adjustment of the set of parameters may include adjusting one of a pulse amplitude, a pulse width, a pulse frequency, relative timing between areas or channels, an ON or OFF timing, stimulation patterns, active electrodes, or fractionalization among active electrodes.

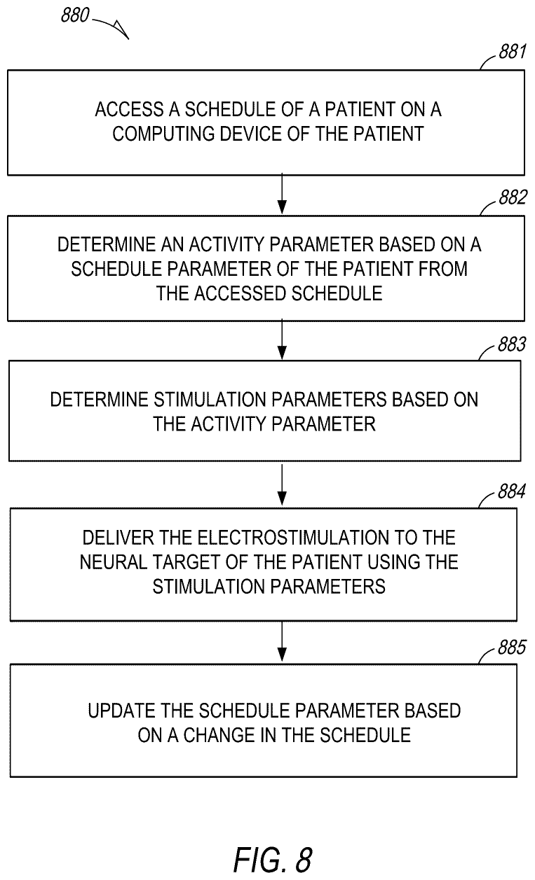

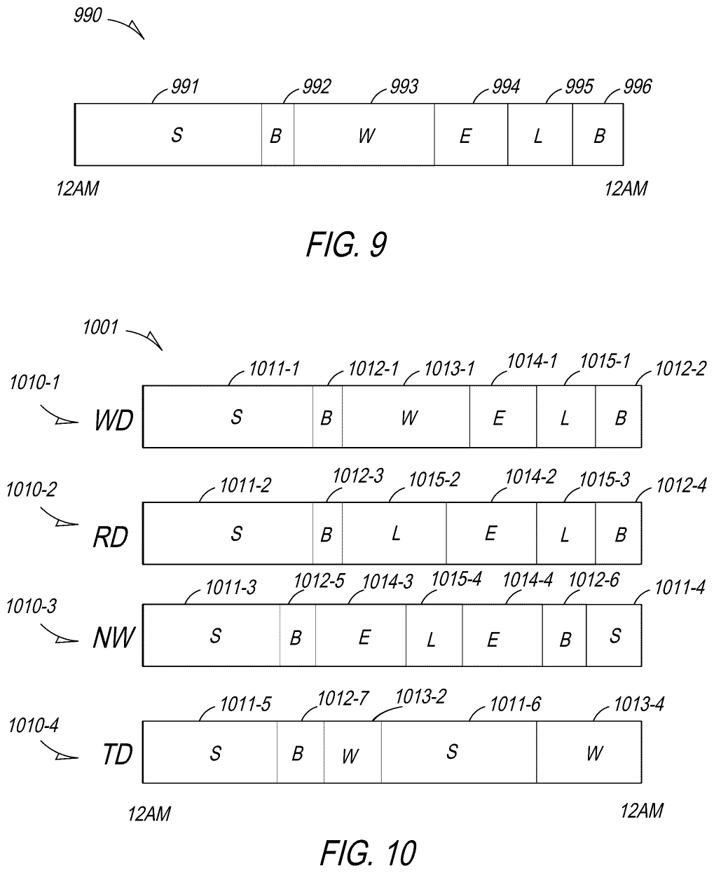

FIG. 8 illustrates, by way of example and not limitation, a method 880 for scheduling neuromodulation therapy. At 881, the method 880 can include accessing a schedule of a patient on a computing device of the patient. The computing device may be a computer, tablet, mobile device, or any other suitable device for processing information. The computing device may be local to the user or may include components that are non-local to the computer. The functions associated with the computing device may be distributed among two or more devices, such that there may be two or more memory devices performing memory functions, two or more processors performing processing functions, two or more displays performing display functions, and/or two or more input devices performing input functions. In some examples, the computing device may include a watch, wristband, smartphone, or the like. The schedule may include an activity parameter associated with a particular time of day and/or associated with a particular day of the week, as will be described further in association with FIGS. 9-11 below.

To retrieve and/or synchronize a calendar from the computing device of the patient, an IPG (such as IPG 102 in FIG. 1) and the computing device could communicate during a regular interval whenever the computing device time registers a schedule change, the IPG time anticipates a schedule change, on a constant (e.g., once per hour, etc.) interval, and/or using other synchronization methods.

At 882, the method 880 may include determining an activity parameter based on a schedule parameter of the patient. The schedule parameter can be associated with a schedule of the patient. The schedule parameter can be an upcoming schedule parameter for delivering the electrostimulation during a subsequent time period. The schedule parameter can include a time of day or a day of a week or a particular time of a month (e.g., the second Saturday, etc.). The activity parameter can be determined based on the time of day and the day of the week. The activity parameter can include at least one of sleeping, reading, working, performing errands, leisure activity, exercising, sitting in a vehicle, and performing daily chores. Each of the activity parameters can be associated with a particular set of stimulation parameters for providing the electrostimulation therapy that is most effective while performing at least one of the activity parameters.

At 883, the method 880 can include determining stimulation parameters based on the activity parameter. Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue; a comparison of a set threshold value; user-determined values or thresholds, or a change/delta from a prior measurement. Each of the electrodes may either be used (an active electrode) or unused (OFF). These and possibly other stimulation parameters taken together comprise a stimulation program that the stimulation circuitry 224 in the IPG 202 of FIG. 2 may execute to provide therapeutic stimulation to a patient.

At 884, the method 880 can include delivering the electrostimulation to the neural target of the patient using the stimulation parameters. An electrostimulator can be used to provide the electrostimulation to the neural target of the patient. A controller circuit can be operably connected to the electrostimulator and be used to perform the steps of method 880.

At 885, the method 880 can include updating the schedule parameter based on a change in the schedule. The change in the schedule can include shifting an activity parameter from occurring at a first time period of a day to a second time period of a day or shifting to occur at a same time of day but during a different day of the week, etc. The change in schedule can be a change from a normal schedule routine. The update to the schedule parameter can include determining a different set of stimulation parameters to use during that schedule parameter and/or during the activity parameter associated with the schedule parameter.

The method 880 may include adjusting a set of stimulation parameters associated with the electrostimulation based on the updated schedule parameter. For example, the set of stimulation parameters may be changed to be used during a different period of time (e.g., for a longer or shorter period of time than originally indicated), during a different portion of the day, different portion of the week, etc. ned state. The adjustment of the set of stimulation parameters may include adjusting one of a pulse amplitude, a pulse width, a pulse frequency, an ON or OFF timing, stimulation patterns, active electrodes, or fractionalization among active electrodes.

FIG. 9 illustrates, by way of example and not limitation, a diagram 990 of activities during a daily schedule of a patient. The diagram 990 illustrates a daily schedule during a 24-hour period from 12 AM to 12 AM. A first period 991 of the day can be associated with an activity parameter including sleep (“S”). A second period 992 of the day can be associated with an activity parameter including using a bathroom (“B”) to prepare for the day or perform other bathroom activities such as brushing, washing, etc. A third period 993 of the day can be associated with an activity parameter including performing work activities (“W”). A fourth period 994 of the day can be associated with an activity parameter including performing errands (“E”). A fifth period 995 of the day can be associated with an activity parameter including performing leisure activities (“L”). A sixth period 996 of the day can be associated with an activity parameter including using a bathroom (“B”) to get ready to go to sleep, such as brushing, bathing or showering, etc.

Each of the activity parameters associated with different periods of the day can correspond to a particular set of stimulation parameters. For each of the schedule parameters, such as for each of the periods of the day, the patient can indicate an activity level (0-10) and a pain score (such as a numerical rating score (NRS)). As an example, the sleep activity during the first period 991 can be associated with an activity level of “0” and an NRS of “2.” Likewise, the second period 992 including using the bathroom can be associated with an activity level of “3” and an NRS of “2”; the third period 993 including performing work activities could be associated with an activity level of “6” and an NRS of “6”; the fourth period 994 including performing errands could be associated with an activity level of “8” and an NRS of “7”; and the fifth period 995 including performing leisure activities could be associated with an activity level of “3” and an NRS of “5.” Each of these inputs could be used to determine the most effective stimulation parameters for each of the associated activity parameters. These activity and/or pain levels can be entered by a patient (e.g., representing a typical day for the patient before the treatment or within a last month or another period of time). The activity and/or pain levels can be detected (e.g., by a wearable device that may include an accelerometer and/or a pain biomarker). Other metrices, e.g., “states,” or sense biopotentials (e.g., ESAP at certain amplitudes, EEG, etc.) may also be incorporated into the decisions.

As an example of how the activity levels and/or pain levels can be incorporated into a decision, the following Example Decision tree is illustrated.

### Example Decision Tree:

```
If Activity < 2 AND Baseline NRS <2
  Schedule = OFF
Else if 2 ≤ Activity < 5 AND 2 ≤ NRS < 5
  Schedule = P1: FAST, 30%, 1 hr ON / 2 hr OFF
Else 2 ≤ Activity < 5 AND NRS ≥ 5
  Schedule = P2: FAST, 30%, 1 hr → 70% 10 min → 2 hr OFF
Else if Activity ≥ 5 AND NRS ≥ 5
  Schedule = P3: Rate Mod, 30%, 2 hr → 70% 10 min → 2 hr OFF
```

FIG. 10 illustrates, by way of example and not limitation, a diagram 1001 of daily schedules that include activities for different types of days 1010-1, 1010-2, 1010-3, 1010-4 of a patient. The types of days 1010-1 to 1010-4 can include a work day (“WD”) 1010-1, a rest day or holiday (“RD”) 1010-2, a non-work or active day (“NW”) 1010-3, and a travel day (“TD”) 1010-4. While a work, rest, non-work, or travel day are described here, examples are not limited and can include any number of types of days that are scheduled and used for neuromodulation therapy. Similar to the description of FIG. 9, the work day 1010-1 can be a daily schedule during a 24-hour period from 12 AM to 12 AM. A first period 1011-1 of the work day 1010-1 can be associated with an activity parameter including sleep (“S”). A second period 1012-1 of the work day 1010-1 can be associated with an activity parameter including using a bathroom (“B”) to prepare for the day or perform other bathroom activities such as brushing, washing, etc. A third period 1013-1 of the work day 1010-1 can be associated with an activity parameter including performing work activities (“W”). A fourth period 1014-1 of the work day 1010-1 can be associated with an activity parameter including performing errands (“E”). A fifth period 1015-1 of the work day 1010-1 can be associated with an activity parameter including performing leisure activities (“L”). A sixth period 1012-2 of the work day 1010-1 can be associated with an activity parameter including using a bathroom (“B”) to get ready to go to sleep, such as brushing, bathing or showering, etc.

Likewise, the rest day 101-2 can include a sleep period 1011-2, a first bathroom period 1012-3, a leisure period 1015-2, an errand period 1014-2, a second leisure period 1015-3, and a second bathroom period 1012-4. Further, the non-work day 1010-3 can include a first sleep period 1011-3, a first bathroom period 1012-5, a first errand period 1014-3, a leisure period 1015-4, a second errand period 1014-4, a second bathroom period 1012-6, and a second sleep period 1011-4. Furthermore, the travel day 1010-4 can include a first sleep period 1011-5, a bathroom period 1012-7, a work period 1013-2, a second sleep period 1011-6 (possibly during a flight or in travel), and a second work period 1013-4 (again, possibly during a flight or in travel).

Each of these corresponding activities (e.g., sleep(S), bathroom (B), work (W), errands (E), leisure (L), etc.) can correspond to their own activity levels and/or pain levels that have an affect on which neuromodulation therapy parameters would provide effective therapy to the patient. Based on the upcoming schedule, the neuromodulation therapy parameters can be set in advance and anticipated prior to the activities occurring. In addition, changes to the schedule and/or activities for each type of day can be made in real-time and can cause changes to the neuromodulation therapy prior to administration of the neuromodulation therapy to the patient.

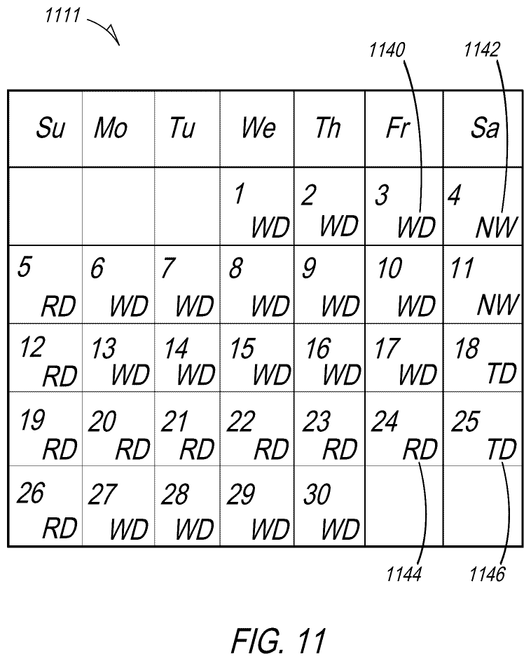

FIG. 11 illustrates, by way of example and not limitation, a diagram of an example of a calendar 1111 showing the different types of days for a patient. The calendar 1111 as illustrated includes 30 days. Each day can be labeled with a type of day that corresponds to a number of activities, as was described in association with FIGS. 9-10. For example, the third (3rd) day of the month in the calendar 1111 can be a work day (WD) 1140, the fourth (4th) day of the month in the calendar 1111 can be a non-work day (“NW”) 1142, a twenty-fourth (24th) day of the month in the calendar 1111 can be a rest day (“RD”) 1144, a twenty-fifth (25th) day of the month in the calendar 1111 can be a travel day (“TD”) 1146, and so forth for each of the days as label in the example of calendar 1111.

A patient and/or a representative of the patient can label or mark each of the calendar days, whether by hand, through a survey, digitally, etc. For example, the patient could manually define programs and intervals for each day “profile,” define just the interval of times, and/or define the programs, while using an AI-assisted (or non-AI) survey to complete and select specific time fields. An example of an AI-assisted question can include: “From when until when is your flight?” Followed by: “Do you intend on sleeping or working during your flight?” Answers would assist in bracketing time intervals of travel programming and then decide if “leisure,” “work,” or “sleep” should be used. Such prompts can help identify which activities are occurring during which time periods in order to determine which corresponding neuromodulation therapy parameters to use.

Several different types of modes can be used to make these determinations. For example, a manual mode can include a user (e.g., a patient, clinician, representative, etc.) manually scheduling programs to be used. An automated mode can include using artificial intelligence (AI) to optimize the program usage by time of day and day of week based on internal and/or external sensing. A hybrid mode can include an application prompting the patient for information about activities scheduled (e.g., sleep, exercise, work, etc.) and outcome measures. AI can also be used to optimize programming usage by time of day, day of week, etc., for a hybrid mode. If the patient's activities appear to deviate from a normal routine, an application can prompt the patient to answer questions to clarify and use new information in optimizing and revising the neuromodulation therapy parameters. In some examples, if an application detects a change in GPS location, an atypical change in GPS location, and/or a time zone change, a prompt can be sent to the patient to answer questions about their reasons for travel (e.g., vacation, work, etc.) and expected activities and use this new information to optimize therapy. The detection of change in GPS location can include a detection of a change in general location data (e.g., cell phone tower data, WiFi triangulation, etc.).

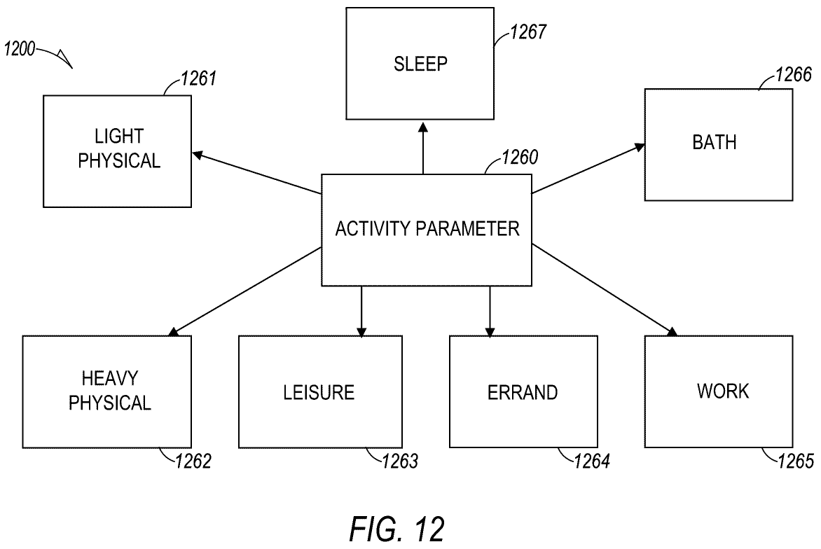

FIG. 12 illustrates, by way of example and not limitation, an example of a number of activity parameters 1260. The number of activity parameters 1260 may include light physical activity 1261, heavy physical activity 1262, leisure activity 1263, performing errands 1264, performing work activities 1265, performing bathroom activities, sleeping, among other actions not described herein. Whether to use each or some of the activity parameters 1260 may be determined based on input from a patient, entries in a calendar or device, etc. For example, when a patient is aware of a scheduled activity or a change in scheduled activity, the patient can enter one or more of the activity parameters 1260 into the schedule to indicate which type of activities the patient will be performing or participating in during a particular period of time.

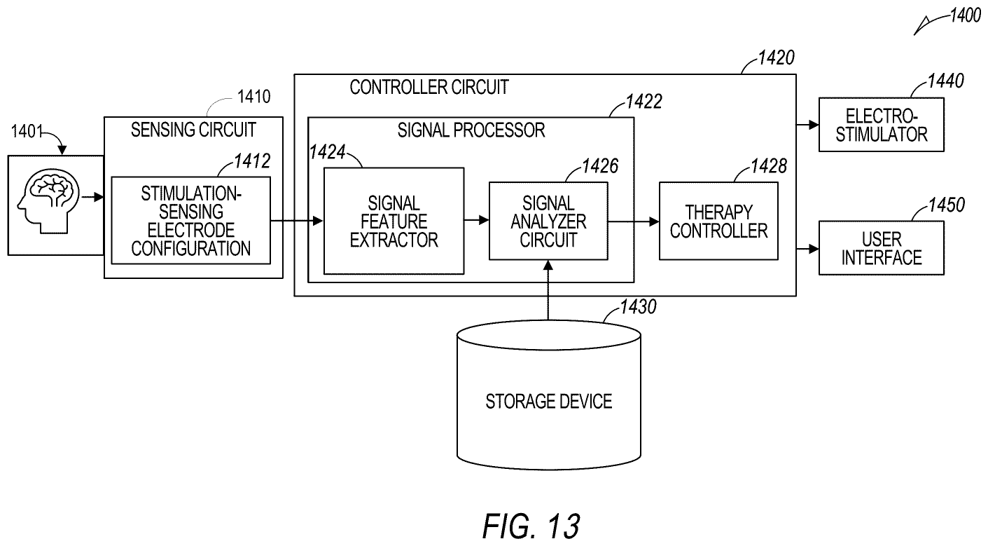

FIG. 13 illustrates, by way of example and not limitation, a neuromodulation therapy system, which may be used to deliver SCS and/or DBS. The system 1400 includes a sensing circuit 1410, a controller circuit 1420, a storage device 1430, an electrostimulator 1440, and a user interface 1450. Portions of the system 1400 may be implemented in the IPG 102 or the CP 104.

The sensing circuit 1410 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 1440. The ring electrodes and/or the segmented electrodes may be configured as sensing electrodes for sensing ERs, or as stimulating electrodes for delivering electrostimulation pulses. The sensing circuit 1410 may sense ERs from one or more sensing electrodes on a lead placed at target issue (e.g., STN) of a patient 1401 in response to electrostimulation pulses delivered from a stimulating electrode at a stimulation site (e.g., a brain target). The ERs may be sensed in accordance with a stimulating-sensing electrode configuration 1412.

The controller circuit 1420 may include circuit sets comprising one or more other circuits or sub-circuits, such as a signal processor 1422 and a therapy controller 1428. The signal processor 1422 may further include a signal feature extractor 1424 and a signal analyzer circuit 1426. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 1420 may be implemented as a part of a microprocessor circuit. The microprocessor circuit may be a dedicated processor such as a digital signal processor, application specific integrated circuit (ASIC), microprocessor, or other type of processor for processing information including physical activity information. Alternatively, the microprocessor circuit may be a general purpose processor that may receive and execute a set of instructions of performing the methods or techniques described herein.

The signal feature extractor 1424 may extract a signal feature from the filtered ER signal (e.g., the ER signal with the stimulation artifact removed). Examples of the signal features may include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, may be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length may be measured as accumulated signal value differences of the sensed evoked response (or an epoch-averaged evoked response) over consecutive unit times (e.g., consecutive data sampling intervals) within the time window. The signal power may be measured as an area under the curve (AUC) of the sensed evoked response (or the epoch-averaged evoked response) within the time window. In some examples, the signal analyzer circuit 1426 may generate a spatial distribution of extracted signal features across sensing locations of the sensing electrodes.

The signal analyzer circuit 1426 may compare the filtered ERs, or signal features or a spatial distribution of signal features derived therefrom, to one or more states or conditions of the patient. In some examples, the states or conditions are used to adjust the neuromodulation parameters. In some examples, the signal analyzer circuit 1426 may accumulate the sensed ERs, and therefore determined states or conditions, obtained in multiple stimulation-ER recording sessions during which stimulation pulses are delivered via a particular stimulating electrode with varying stimulation parameter settings (e.g., stimulation amplitude, frequency, or pulse width), and compare the accumulated ERs (or signal features or a spatial distribution of signal features derived from the filtered ERs) to previously stored states or conditions. In an example, the states or conditions of the patient may be user-provided or loaded into the system for easy recognition. In another example, the state or condition may be associated with a target ER or target ER feature template representing a patient-specific ER feature or a population-based ER feature to electrostimulation of the neural target. In an example, the target ER template may be used to adjust the neuromodulation therapy to relieve symptoms or other goals such as co-therapy (e.g., leads that inject drugs or light), and side-effect avoidance. The signal analyzer circuit 1426 may determine a distribution of sensed ER features, compare the determined distribution of sensed ERs to the corresponding states or conditions to determine whether to adjust the neuromodulation therapy.

The therapy controller 1428 may generate a control signal to the electrostimulator 1440 to adjust the thresholds of the neuromodulation therapy based on the determined state or condition. The electrostimulator 1440 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 1428 may be implemented as a proportional integral (PI) controller, a proportional-integral-derivative (PID) controller, or other suitable controller that takes the comparison of the sensed ERs (or features or a distribution of the features thereof) to the corresponding states or conditions as a feedback on the adjustment of stimulation settings.

The electrostimulator 1440 may be an implantable module, such as incorporated within the IPG 102 in FIG. 1. Alternatively, the electrostimulator 1440 may be an external stimulation device, such as incorporated with the ETS 40. In some examples, the user may choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 1440 may deliver stimulation in accordance with the adjusted therapy setting.

In some examples, the therapy controller 1428 may generate a recommendation to the user to adjust the device setting (e.g., a programmable parameter of the electrostimulator 1440) to cause the sensed ERs to align with or to compare more favorably to one or more states or conditions of the patient. In some embodiments, the display may provide a suggestion to the user to adjust stimulation parameters to cause the since developed responses to more favorably compare to the state or condition or a transition to a different state or condition. In some examples, the therapy controller 1428 may determine or modify therapeutic stimulation settings based on the sense ERs or features thereof and the corresponding determined state or condition of the patient. The electrostimulator 1440 may deliver therapeutic stimulation (e.g., DBS) in accordance with the determined or modified therapeutic stimulation settings.

In some examples, the user interface 1450 allows a physician to remotely review therapy settings and treatment history, consult with the patient to obtain information including pain relief and SCS-related side effects or symptoms, perform remote programming of the electrostimulator 1440, or provide other treatment options to the patient. The user interface 1450 may allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program may include a set of stimulation parameters with respective pre-determined values. In some examples, the user interface 1450 may include a display to display textually or graphically information provided by the user via an input unit, and device settings including, for example, feature selection, sensing configurations, signal pre-processing settings, therapy settings, optionally with any intermediate calculations. In an example, the user interface 1450 may present to the user an “optimal” or improved therapy setting, such as determined based on a closed-loop or adaptive feedback control of electrostimulation based on a selected evoked response signal feature, in accordance with various embodiments discussed in this document. In some examples, the user may use the user interface 1450 to provide feedback on a neuromodulation therapy, including, for example, side effects or symptoms arise or persist associated with the neurostimulation, or severity of the symptom or a side effect.

As used herein, the term “machine readable medium” may include any medium that is capable of storing, encoding, or carrying instructions for execution by a processing device or machine and that causes the processing device or machine to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
determining an activity parameter associated with providing electrostimulation to a neural target of a patient based on a schedule parameter associated with the patient, wherein the activity parameter is determined based on received activity data from the patient;
determining stimulation parameters based on the activity parameter; and
delivering the electrostimulation to the neural target of the patient via a stimulating electrode using the stimulation parameters.

2. The method of claim 1, wherein the schedule parameter is determined based on a schedule of the patient.

3. The method of claim 2, wherein the schedule parameter is determined by accessing the schedule on a computing device of the patient.

4. The method of claim 2, comprising updating the schedule parameter based on a change in the schedule.

5. The method of claim 1, wherein the activity data is received from the patient in response to sending a request to the patient for the activity data.

6. The method of claim 1, wherein the schedule parameter is an upcoming schedule parameter for delivering the electrostimulation during a subsequent time period.

7. The method of claim 1, comprising analyzing the activity parameter and the time of day and the day of the week associated with the activity parameter to determine whether the activity parameter is a deviation from a normal routine of the patient.

8. The method of claim 7, comprising requesting additional data from the patient in response to determining that the activity parameter deviates from the normal routine.

9. The method of claim 1, wherein determining stimulation parameters comprises determining a period of time to deliver the electrostimulation using the stimulation parameters.

10. The method of claim 9, wherein the period of time is determined based on a corresponding period of time that the activity parameter is associated with.

11. The method of claim 1, wherein determining the stimulation parameters comprises:
determining a first set of stimulation parameters used to deliver the electrostimulation during a first period of time; and
determining a second set of stimulation parameters used to deliver the electrostimulation during a second period of time.

12. The method of claim 11, wherein the first period of time is associated with a first activity parameter and the second period of time is associated with a second activity parameter.

13. The method of claim 1, comprising determining a therapy satisfaction experienced by the patient during a period of time.

14. The method of claim 13, wherein the stimulation parameters are determined using the determined pain level as an input.

15. The method of claim 13, wherein the pain level is determined by receiving pain data from the patient.

16. The method of claim 15, wherein the pain data is received in response to sending a request to the patient to indicate the pain level.

17. A non-transitory computer-readable medium storing instructions executable by a processor to:
determine an activity parameter and a schedule parameter associated with providing electrostimulation to a neural target of a patient by accessing a schedule of the patient;
determine stimulation parameters based on the activity parameter; and
deliver the electrostimulation to the neural target of the patient via a stimulating electrode using the stimulation parameters.

18. The non-transitory computer-readable medium of claim 17, wherein the instructions are executable by the processor to determine a therapy satisfaction at the time of day and the day of the week for the patient.

19. The non-transitory computer-readable medium of claim 17, wherein the instructions are executable by the processor to determine the stimulation parameters such that a prior electrostimulation delivered during a similar activity at a similar time and day of week that achieved a particular therapy level is scheduled to be delivered during the similar activity when anticipated to be performed at a later period of time.

20. A system, comprising:
an electrostimulator configured to provide electrostimulation to a neural target of a patient; and
a controller circuit operably connected to the electrostimulator, the controller circuit configured to:
determine an activity parameter based on a schedule parameter of the patient, wherein the schedule parameter is associated with a schedule of the patient;
determine stimulation parameters based on the activity parameter; and
deliver the electrostimulation to the neural target of the patient using the stimulation parameters.
